(19) United States
(12) Patent Application Publication
(10) Pub. No.: US 2023/0248289 A1
(43) Pub. Date: Aug. 10, 2023

(54) FASTENING DEVICE FOR TEMPORARILY FASTENING A PORTABLE SENSOR DEVICE TO A HUMAN BODY

(71) Applicant: COALA-LIFE AB, Uppsala (SE)

(72) Inventors: Amanda TULKKI, Uppsala (SE); Magnus SAMUELSSON, Uppsala (SE)

(21) Appl. No.: 18/001,301

(22) PCT Filed: Jun. 7, 2021
(86) PCT No.: PCT/EP2021/065166

§ 371 Date: Dec. 9, 2022

(30) Foreign Application Priority Data
Jun. 11, 2020 (SE) 2050692-9

(51) Int. Cl. A61B 5/252; A61B 5/256

(52) U.S. Cl. CPC A61B 5/252; A61B 5/256

(57) ABSTRACT

It is provided a fastening device for temporarily fastening a portable sensor device to a human body, the fastening device comprising: a device holder for releasably holding a portable sensor device configured to capture electrocardiogram signals and audio signals; a skin sealer configured to form an essentially airtight interface with a human body; and a bellows provided between the skin sealer and the device holder, the bellows, configured to fasten the fastening device to the human body using a suction effect after a portable sensor device held in the device holder is pushed towards the human body to let air escape from the bellows via the skin sealer.

### TECHNICAL FIELD

The invention relates to a fastening device and corresponding kit for temporarily fastening a portable sensor device to a human body.

### BACKGROUND

ECG is an established technology where electric signals generated by the body of a patient are measured and analysed. Traditionally, a number of electrodes are placed on the body at various places. A conductive gel is used to provide better conductive contact between the electrodes and the skin. The patient typically lies down for minutes when the ECG is taken. The data detected using the electrodes is recorded and can be analysed by a professional, such as a physician or trained nurse. Once the measurement procedure is done, the conductive gel is wiped off.

While having proved useful, the traditional way of obtaining an ECG is not optimal in all cases. For instance, such an ECG needs to be measured in a clinic and the procedure is messy for the patient.

Lately, portable sensor devices with integral electrodes for obtaining ECG data have been developed. These portable sensor devices allow users to capture ECG data at will, and also without the use of conductive gel. This gives the user greater control over when to capture ECG data and also in a much more convenient and less messy way. Such portable sensor devices can also be configured to measure phonocardiogram (PCG) data, i.e. sound data of the heart.

In order to allow the portable sensor device to capture its measurement without the user having to constantly hold it in place, a fastening device can be provided. Fastening devices in the prior art can e.g. be based on adhesives. However, the adhesives are not always reliable. Moreover, a significant proportion of the population have or develop allergies to such adhesives, preventing such fastening devices from being a universal solution.

### SUMMARY

It is an object to provide a fastening device, for temporarily fastening a portable sensor device to a human body, which is more convenient than what is known in the prior art.

According to a first aspect, it is provided a fastening device for temporarily fastening a portable sensor device to a human body, the fastening device comprising: a device holder for releasably holding a portable sensor device being configured to capture electrocardiogram signals and audio signals; a skin sealer configured to form an essentially airtight interface with a human body; and a bellows provided between the skin sealer and the device holder, the bellows, configured to fasten the fastening device to the human body using a suction effect after a portable sensor device held in the device holder is pushed towards the human body to let air escape from the bellows via the skin sealer.

The device holder may comprise an opening, in which case the sides of the opening are configured form a substantially airtight interface with a portable sensor device.

The device holder may comprise an airtight membrane configured to separate the portable sensor device and the human body.

At least part of the bellows may be configured to be provided between the portable sensor device and the body, when the fastening device is in use.

The fastening device may further comprise an air pressure sensor provided on an inside of the fastening device, whereby the fastening device is configured to detect when there is poor suction effect when the fastening device is in contact with the human body.

The device holder may comprise a snap fastener.

According to a second aspect, it is provided a sensor kit comprising the fastening device according to the first aspect and a portable sensor device to be located in the device holder of the fastening device.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

Aspects and embodiments are now described, by way of example, with reference to the accompanying drawings, in which:

FIGS. 1A-B are schematic diagrams illustrating an environment in which embodiments presented herein can be applied;

FIG. 2 is a schematic diagram of a bottom view illustrating a physical representation of the portable sensor device of FIG. 1 according to one embodiment;

FIGS. 3A-B are schematic diagrams illustrating the fastening device of FIG. 1 according to one embodiment; and

FIGS. 4A-B are schematic perspective views illustrating embodiments of fastening devices holding respective portable sensor devices.

### DETAILED DESCRIPTION

The aspects of the present disclosure will now be described more fully hereinafter with reference to the accompanying drawings, in which certain embodiments of the invention are shown. These aspects may, however, be embodied in many different forms and should not be construed as limiting; rather, these embodiments are provided by way of example so that this disclosure will be thorough and complete, and to fully convey the scope of all aspects of invention to those skilled in the art. Like numbers refer to like elements throughout the description.

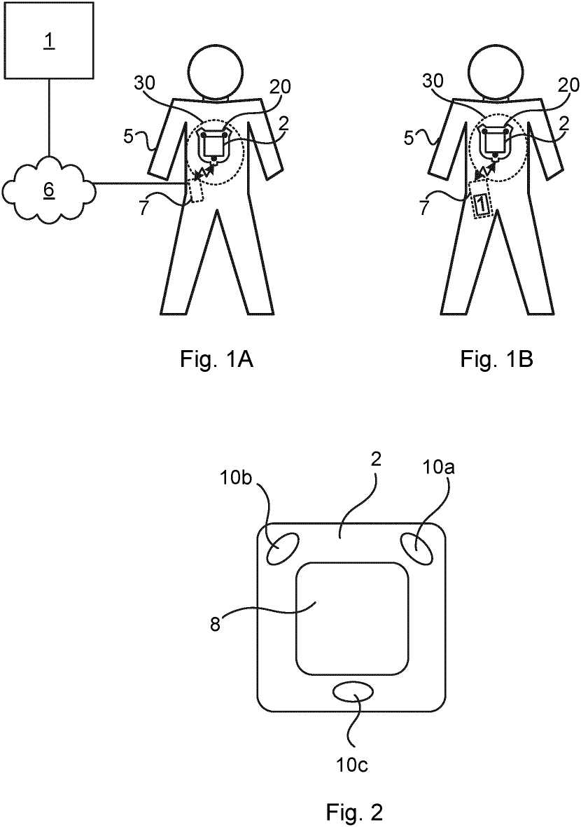

FIGS. 1A-B are schematic diagrams illustrating an environment in which embodiments presented herein can be applied.

Looking first to FIG. 1A, it is here shown a user 5 having a portable sensor device 2 provided in a fastening device 20. The fastening device 20 comprises a bellows and is fastened to the user 5, more specifically to the chest of the user 5, using a suction effect after air is pushed out of the bellows.

The user 5 also carries a smartphone 7 e.g. in a pocket. The portable sensor device 2 and the smartphone 7 can communicate over any suitable wireless interface, e.g. using Bluetooth or Bluetooth Low Energy (BLE), ZigBee, any of the IEEE 802.11× standards (also known as WiFi), etc.

The smartphone 7 is also connected to a wide area network 6, such as the Internet, e.g. via WiFi or a cellular network, to allow communication with an analysis device 1, here in the form of a server. The portable sensor device 2 captures ECG data and PCG data, via the fastening device 20 and sends this data, via the smartphone 7, to the analysis device 1. This allows the analysis device 1 to determine whether the heart of the user 5 can be considered to be in a normal state or whether the heart needs further examination based on the PCG data and the ECG data captured by the portable sensor device 2. Further investigation can be determined to be needed e.g. if any abnormal heart condition cannot be ruled out. It is to be noted that even if further investigation is to be performed, the heart can in fact be normal, i.e. non-pathological.

Together, the fastening device 20 and the portable sensor device 2 is denoted a sensor kit 30.

In FIG. 1B, the smartphone 7 contains the analysis device 1. In this way, the analysis can be performed locally, without the need for immediate access to the wide area network.

FIG. 2 is a schematic diagram of a bottom view illustrating a physical representation of the portable sensor device 2 of FIG. 1 according to one embodiment. The portable sensor device 2 comprises here a first electrode 10a, a second electrode 10b and a third electrode 10c. In order to capture the ECG data, the electrodes 10a-c need to make electrical contact with the body of the user. As explained in further detail below, the electrodes 10a-c make contact with the body when the portable sensor device 2 is placed in the fastening device and the portable sensor device 2 is pressed towards the body. Using the electrodes, one or more analogue ECG signals are captured. The analogue ECG signals are converted to digital ECG signals using an analogue to digital (A/D) converter. The digital ECG signal is then sent to the analysis device.

An audio sensor 8, e.g. in the form of a microphone, is provided to convert sound waves originating from the body into electric analogue PCG signals. The analogue PCG signals are converted to digital PCG signals using an A/D converter. The digital PCG signal is then sent to the analysis device for analysis together with the ECG signal.

It is to be noted that the portable sensor device 2 may also optionally comprise one or more user interface elements (not shown), e.g. push buttons, Light Emitting Diodes (LEDs), a display, a speaker, a user microphone, etc.

It is to be noted that the portable sensor device 2 could also be provided with two, four or any other suitable number of electrodes, as long as there are at least two electrodes.

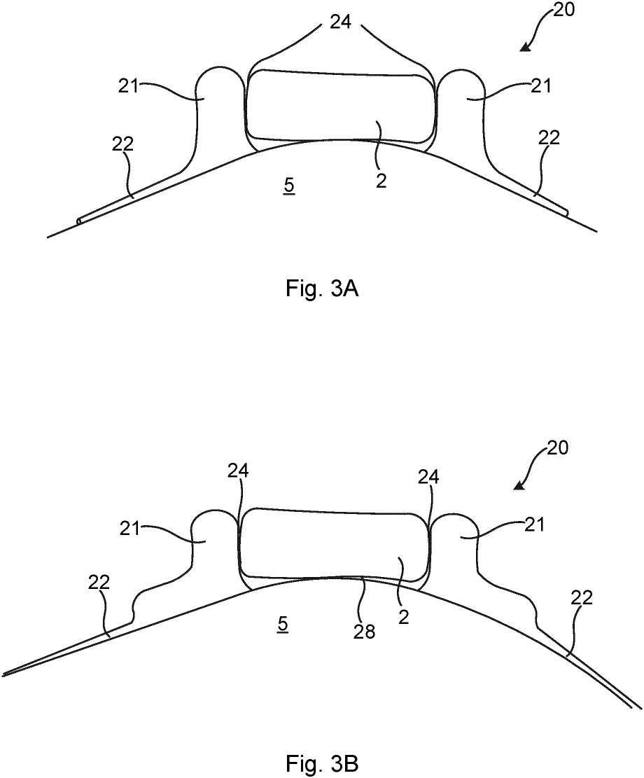

FIGS. 3A-B are schematic cross-section diagrams illustrating the fastening device 20 of FIG. 1 according to various embodiments. The fastening device 20 is used for temporarily fastening a portable sensor device to a human body.

A device holder 24 is provided for releasably holding a portable sensor device 2. As explained above, the portable sensor device 2 is configured to capture ECG signals and audio (PCG) signals. In one embodiment, the device holder 24 comprises one or more snap fastener to keep the portable sensor device 2 in place. In any case, the device holder 24 is adapted to match and receive a portable sensor device 2 of a particular geometric configuration.

A skin sealer 22 is configured to form an essentially airtight interface with a human body. Essentially airtight is to be construed as sufficiently airtight for fastening the portable sensor device to the human body using a suction effect during an intended period of obtaining measurements from the sensor device.

A bellows 21 is provided between the skin sealer 22 and the device holder 24. The bellows 21 is configured to fasten the fastening device 20 to the human body using a suction effect. This is achieved when the portable sensor device 2 held in the device holder 24 is pushed 27 towards the human body, which lets air escape from the bellows 21 via the skin sealer 22. In other words, the pushing action 27 increases air pressure inside the bellows to a point that the skin sealer 22 temporarily releases from the body, to thereby let air escape. When the pushing ends, the elastic resilience of the bellows 21 reduces air pressure inside the bellows, causing a suction effect. The skin sealer 22 is sufficiently airtight in its interface with the body to keep any significant amount of air to pass into the bellows between the skin sealer 22 and the body, whereby the fastening device, and its portable sensor device 2 is kept fastened to the body. Moreover, the suction effect pulls the skin up, towards the portable sensor device 2, further improving the conductive connection between the portable sensor device and the skin. To remove the fastening device, the user simply lifts up the skin sealer to let air into the bellows, to thereby break the suction effect. No other fastening means is necessary, such as a strap or adhesive; making the fastening convenient and comfortable for the user.

In one embodiment, as illustrated in FIG. 3A, the device holder 24 comprises an opening. The opening is configured to receive the portable sensor device. The sides of the opening are configured form a substantially airtight interface with a portable sensor device 2. In this embodiment, the portable sensor device 2 is configured such that it is airtight between the side which is on the inside of the device holder 24 and the side which is on the outside of the device holder 24.

In one embodiment, as illustrated in FIG. 3B, the device holder 24 comprises an airtight membrane configured to separate the portable sensor device and the human body. In this embodiment, the portable sensor device 2 does not need to be airtight. However, in order to allow the portable sensor device to measure ECG, the membrane 28 comprises electrically conductive sections, at least as many as there are electrodes in the portable sensor device. The electrically conductive sections are conductively separated from each other by airtight insulator sections, to avoid shortcutting the electrodes of the portable sensor device.

Optionally, the fastening device 20 further comprises an air pressure sensor. The air pressure sensor can be used to detect when there is poor suction effect with the human body. In this way, the user can be alerted if the suction effect deteriorates and can address the situation, e.g. by pushing the portable sensor device towards the skin.

The fastening device 20 can be reusable. For instance, the fastening device 20 can be made completely or partly out of silicone, rubber or other rubber like polymer which forms a substantially airtight seal with the body and portable sensor device 2. The material of the fastening device 20, or at least of the skin sealer 22, is of a low-allergy material, such as silicone.

In one embodiment, the fastening device 20 is manufactured in one piece to avoid any seams between different part of the fastening device. Hence, the different parts of the fastening device mentioned here, such as the bellows 21, the skin sealer 22 and the device holder 24, do not need to be separately manufactured and joined; these parts can be construed as different functional parts of a single fastening device 20. Nevertheless, it is also possible that the different parts 21, 22, 24 of the fastening device are indeed manufactured separately and assembled to make up the fastening device 20.

The fastening device 20 and the portable sensor device 2 of FIG. 1 are thus used together to capture measurements for ECG and for PCG. In order to capture measurements for ECG and for PCG, the portable sensor device 2 is placed inside the fastening device 20. The fastening device 20 is then placed on the skin body of the user, e.g. on the chest of the user, close to the heart, and fastened using the suction effect as mentioned above.

The user can then cover the fastening device 20 and the portable sensor device 2, e.g. by wearing covering clothes. In this way, the user can perform any activity while ECG and PCG measurements are captured. For instance, measurements can be captured in the daily life of the user, giving much more valuable data and covering a longer time period than if the user has to actively measure each time.

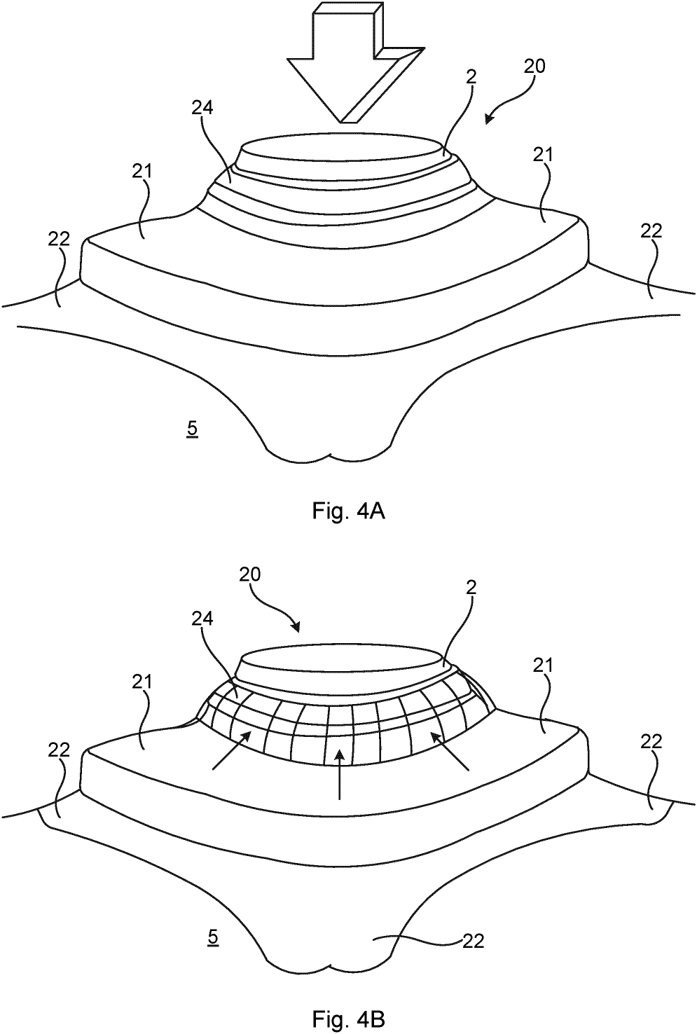

FIGS. 4A-B are schematic perspective views illustrating embodiments of fastening devices holding respective portable sensor devices.

The embodiment shown in FIG. 4A corresponds to the embodiment shown in FIG. 3A, where the portable sensor device 2 is inserted in an opening of the device holder. As for the embodiment of FIG. 3A, the bellows 21 is here configured primarily for lateral expansion (perpendicular to the direction of the pushing force 27) when the pushing force is applied.

The embodiment shown in FIG. 4B corresponds to the embodiment shown in FIG. 3B, where a membrane is provided between the bellows and the portable sensor device. Alternatively, at least part of the bellows 21 can be configured to be provided between the portable sensor device 2 and the body 5, when the fastening device 20 is in use. This allows the portable sensor device 2 to be more distal from the body 5. As for the embodiment of FIG. 3B the bellows 21 is here configured primarily for expansion in a reverse direction to the direction of the pushing force 27, when the pushing force 27 is applied. Specifically, the outer parts of the bellows 21 expand away from the body 5 when the pushing force 27 is applied.

The aspects of the present disclosure have mainly been described above with reference to a few embodiments. However, as is readily appreciated by a person skilled in the art, other embodiments than the ones disclosed above are equally possible within the scope of the invention, as defined by the appended patent claims. Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A fastening device for temporarily fastening a portable sensor device to a human body, the fastening device comprising:
a device holder for releasably holding a portable sensor device configured to capture electrocardiogram signals and audio signals;
a skin sealer configured to form an essentially airtight interface with a human body; and
a bellows provided between the skin sealer and the device holder, the bellows being configured to fasten the fastening device to the human body using only a suction effect after a portable sensor device held in the device holder is pushed towards the human body to let air escape from the bellows via the skin sealer.

2. The fastening device according to claim 1, wherein the device holder comprises an opening and wherein the sides of the opening are configured form a substantially airtight interface with a portable sensor device.

3. The fastening device according to claim 1, wherein the device holder comprises an airtight membrane configured to separate the portable sensor device and the human body.

4. The fastening device according to claim 1, wherein at least part of the bellows is configured to be provided between the portable sensor device and the body, when the fastening device is in use.

5. The fastening device according to claim 1, further comprising an air pressure sensor provided on an inside of the fastening device, whereby the fastening device is configured to detect when there is poor suction effect when the fastening device is in contact with the human body.

6. The fastening device according to claim 1, wherein the device holder comprises a snap fastener.

7. A sensor kit comprising the fastening device according to claim 1 and a portable sensor device to be located in the device holder of the fastening device.
